(19) United States
(12) Patent Application Publication
(10) Pub. No.: US 2026/0174928 A1
(43) Pub. Date: Jun. 25, 2026

(54) DRESSING FOR PROMOTING WOUND HEALING AND METHOD FOR MANUFACTURING THE SAME

(71) Applicant: YUJIAN International Biotechnology, Inc., Hsinchu City (TW)

(72) Inventors: YAO-JEN TU, Hsinchu City (TW); YU-SHAN LIU, Hsinchu City (TW); HUI-LAN KUO, Hsinchu City (TW); CHUN-LAN LO, Hsinchu City (TW); CHI-WEI KUO, Hsinchu City (TW); YI -LING YANG, Hsinchu City (TW); CHIN-CHANG YANG, Hsinchu City (TW)

(21) Appl. No.: 19/419,407

(22) Filed: Dec. 15, 2025

Related U.S. Application Data
(60) Provisional application No. 63/738,634, filed on Dec. 24, 2024.

(51) Int. Cl. A61L 26/00; A61K 31/706; A61K 31/728; A61K 36/898; A61P 17/02

(52) U.S. Cl. CPC A61L 26/0066; A61K 31/706; A61K 31/728; A61K 36/898; A61P 17/02

(57) ABSTRACT

A method for manufacturing a dressing that promotes wound healing comprises at least the following steps: providing a container; adding purified water to the container; adding a first active ingredient to the container, wherein the first active ingredient is low-molecular-weight hyaluronic acid having a molecular weight of not more than 500,000 Daltons, and which accounts for 0.01% to 1% by weight of the entire dressing; and adding a second active ingredient to the container, wherein the second active ingredient is β-nicotinamide mononucleotide and accounts for 0.01% to 1% by weight of the entire dressing; or the second active ingredient is a plant stem cell extract and accounts for 0.1% to 5% by weight of the entire dressing.

### CROSS-REFERENCE TO RELATED APPLICATION

Pursuant to 35 U.S.C. § 119(a), this application claims the benefit of the priority to USA Provisional Patent Application No. 63/738,634 filed on Dec. 24, 2024. The contents of the prior application are incorporated herein by reference in their entirety.

### BACKGROUND

### Technical Field

The present disclosure relates to a dressing for promoting wound healing and a method for manufacturing the same, more specifically to, a dressing having active ingredients such as low-molecular-weight hyaluronic acid (HA) and one or more of 0-nicotinamide mononucleotide (β-NMN) and a plant stem cell extract, and a method for manufacturing the same.

### Related Art

The skin is the largest organ in the human body, performing multiple functions, including barrier, defense, and regulation. Consequently, damage to it not only affects appearance but can also lead to infection and systemic complications. Wound healing is a highly dynamic, multi-stage physiological process, sequentially encompassing hemostasis, inflammation, proliferation, and tissue remodeling, each involving the coordinated action of different cell populations and cytokines. However, regarding wounds caused in clinical scenarios such as chronic wounds (e.g., diabetic foot ulcers, venous ulcers, and pressure ulcers) and acute wounds (e.g., car accident wounds, knife wounds, falls and abrasions, and surgical wounds), these processes are often delayed or interrupted. Epidemiological data show that tens of millions of patients are affected worldwide, placing a heavy burden on healthcare systems.

Compared to dry dressings, wet dressings offer an ideal microenvironment, thus improving the wound healing process. However, clinical treatment remains challenging, including inadequate infection control in chronic wounds, scar formation, and high heterogeneity among patients. Therefore, the industry continues to strive to improve wet dressings, i.e., developing a wet dressing that combines protective properties with the ability to promote angiogenesis and regeneration. Specifically, the wet dressing can significantly reduce inflammation, accelerate epidermal coverage and enhance overall repair in the early and middle stage of the healing process.

### SUMMARY

An object of the present disclosure is to provide a dressing that overcomes the limitations of the prior art, specifically a dressing using purified water as a solvent, thereby suitable for use as a wet dressing. In addition, the dressing can be modified by incorporating a thickening agent to form a gel consistency, or an emulsifying agent to form an emulsion consistency. Furthermore, the dressing comprises at least two active ingredients. The first active ingredient is low-molecular-weight hyaluronic acid (HA), and the second active ingredient is selected from β-nicotinamide mononucleotide (β-NMN) or a plant stem cell extract. Consequently, the dressing can exhibit anti-inflammatory and healing-accelerating effects in the early stages of the wound healing process.

In accordance with one object of the present disclosure, an embodiment of the present disclosure provides a dressing for promoting wound healing. The dressing uses purified water as a solvent and comprises a first active ingredient and a second active ingredient. The first active ingredient is low-molecular-weight hyaluronic acid (HA) having a molecular weight of not more than 500,000 Daltons and accounting for 0.01% to 1% by weight of the entire dressing. The second active ingredient is β-nicotinamide mononucleotide (β-NMN) and accounts for 0.01% to 1% by weight of the entire dressing; or the second active ingredient is a plant stem cell extract and accounts for 0.1% to 5% by weight of the entire dressing.

Optionally, the dressing for promoting wound healing further comprises an osmotic pressure adjusting agent selected from one or more of chloride salts and lactate salts, which accounts for 0.4% to 1% by weight of the entire dressing.

Optionally, the dressing for promoting wound healing further comprises a solubilizing and film-forming moisturizing agent which accounts for not more than 8% by weight of the entire dressing.

Optionally, the solubilizing and film-forming moisturizing agent comprises one or more of propylene glycol (PG), glycerin, dipropylene glycol (DPG), and 1,3-propanediol (PDO).

Optionally, the solubilizing and film-forming moisturizing agent consists essentially of propylene glycol and glycerin, wherein the propylene glycol accounts for 1% to 3% by weight of the entire dressing, and the glycerin accounts for 1% to 3% by weight of the entire dressing.

Optionally, the dressing for promoting wound healing further comprises a preservative agent accounting for not more than 0.3% by weight of the entire dressing.

Optionally, the preservative agent is methylparaben (MP) or ethylparaben (EP) and accounts for 0.05% to 0.2% by weight of the entire dressing.

Optionally, the osmotic pressure adjusting agent is sodium chloride.

Optionally, the second active ingredient is β-nicotinamide mononucleotide, and the dressing further comprises a third active ingredient, namely, a plant stem cell extract, which accounts for not more than 5% by weight of the entire dressing.

Optionally, the plant stem cell extract is an orchid plant stem cell extract.

Optionally, the dressing for promoting wound healing further comprises a pH-adjusting agent for adjusting a pH value of the dressing to between 5.5 and 9.

Optionally, the pH-adjusting agent is phosphates.

Optionally, the dressing is in an aqueous liquid form.

Optionally, the dressing for promoting wound healing further comprises a thickening agent for rendering the dressing in a gel form.

Optionally, the thickening agent is selected from the group consisting of xanthan gum, hydroxyethylcellulose (HEC) gum, high-molecular-weight hyaluronic acid, and combinations thereof.

Optionally, the dressing for promoting wound healing further comprises an emulsifying agent for rendering the dressing in an emulsion form.

Optionally, the emulsifying agent is selected from the group consisting of polysorbate (TWEEN), sorbitan laurate (SPAN), mineral oil, vegetable oil, and combinations thereof.

In accordance with one object of the present disclosure, an embodiment of the present disclosure provides a method for manufacturing a dressing that promotes wound healing. The method comprises steps of: providing a first container; adding purified water into the first container; adding a first active ingredient to the first container, wherein the first active ingredient is low-molecular-weight hyaluronic acid having a molecular weight of not more than 500,000 Daltons and accounting for 0.01% to 1% by weight of the entire dressing; and adding a second active ingredient to the first container, wherein the second active ingredient is β-nicotinamide mononucleotide and accounts for 0.01% to 1% by weight of the entire dressing, or the second active ingredient is a plant stem cell extract and accounts for 0.1% to 5% by weight of the entire dressing.

Optionally, the method further comprises a step of: adding an osmotic pressure adjusting agent into the first container, wherein the osmotic pressure adjusting agent is selected from one or more of chloride salts and lactate salts and accounts for 0.4% to 1% by weight of the entire dressing.

Optionally, the method further comprises a step of: adding a preservative agent into the first container, wherein the preservative agent accounts for not more than 0.3% by weight of the entire dressing.

Optionally, the method further comprises a step of: adding a solubilizing and film-forming moisturizing agent into the first container, wherein the solubilizing and film-forming moisturizing agent accounts for not more than 8% by weight of the entire dressing.

Optionally, the dressing is in an aqueous liquid form, and the method further comprises a step of: filtering and sterilizing liquid in the first container, and then canning the liquid.

Optionally, the method further comprises a step of: adding a pH-adjusting agent into the first container, and adjusting a pH value of the dressing to between 5.5 and 9.

Optionally, the second active ingredient is β-nicotinamide mononucleotide, and the method further comprises a step of: adding a third active ingredient into the first container, wherein the third active ingredient is a plant stem cell extract and accounts for not more than 5% by weight of the entire dressing

Optionally, the method further comprises a step of: adding a thickening agent into the first container to render the dressing in a gel form.

Optionally, the method further comprises steps of: adding an emulsifying agent into a second container; transferring liquid in the first container into the second container; and emulsifying mixture liquid in the second container to render the dressing in an emulsion form.

To sum up, the present disclosure provides a dressing and a method for manufacturing the same. The dressing is capable of exhibiting anti-inflammatory effects and accelerated healing effects in the early stages of the wound healing process.

### BRIEF DESCRIPTION OF DRAWINGS

FIG. 1 is a flow chart of a method for manufacturing a dressing that promotes wound healing according to a first embodiment of the present disclosure.

FIG. 2 is a flow chart of a method for manufacturing a dressing that promotes wound healing according to a second embodiment of the present disclosure.

FIG. 3 is a flow chart of a method for manufacturing a dressing that promotes wound healing according to a third embodiment of the present disclosure.

FIG. 4 is a flow chart of a method for manufacturing a dressing that promotes wound healing according to a fourth embodiment of the present disclosure.

FIG. 5 is a flow chart of a method for manufacturing a dressing that promotes wound healing according to a fifth embodiment of the present disclosure.

FIG. 6 shows a histogram of wound healing rate in a cell experiment using the dressing that promotes wound healing provided by the present disclosure.

FIG. 7A shows sequential wound images from Day 0 to Day 7 in animal experiments using different dressings to promote wound healing.

FIG. 7B shows sequential wound images from Day 8 to Day 14 of an animal experiment using different dressings to promote wound healing.

FIG. 8 is a line graph showing the ratios of wound areas from Day 0 to Day 14 in animal experiments on wound healing using different dressings.

FIG. 9 shows microscopic images of skin tissue at 1 and 2 weeks post-surgery in animal experiments using different dressings for wound healing, wherein a portion of the images are magnified to observe inflammation.

FIG. 10 shows microscopic images of skin tissue at 1, 2, and 6 weeks post-surgery in animal experiments using different dressings to promote wound healing.

### DETAILS OF EXEMPLARY EMBODIMENTS

The present disclosure is directed to providing a dressing capable of exhibiting anti-inflammatory effects and accelerated healing effects in the early stages of the healing timeline. The wound healing-promoting dressing uses purified water as a solvent and comprises a first active ingredient and a second active ingredient. The first active ingredient is a low-molecular-weight hyaluronic acid (HA), a molecular weight of low-molecular-weight HA is not more than 500,000 Daltons, and the low-molecular-weight HA accounts for 0.01 to 1% by weight of the entire dressing. The second active ingredient is β-nicotinamide mononucleotide (β-NMN) and accounts for 0.01 to 1% by weight of the entire dressing, or is a plant stem cell extract and accounts for 0.1 to 5% by weight of the entire dressing. For the purpose of facilitating understanding of the present disclosure, the following embodiments are described in conjunction with the accompanying drawings.

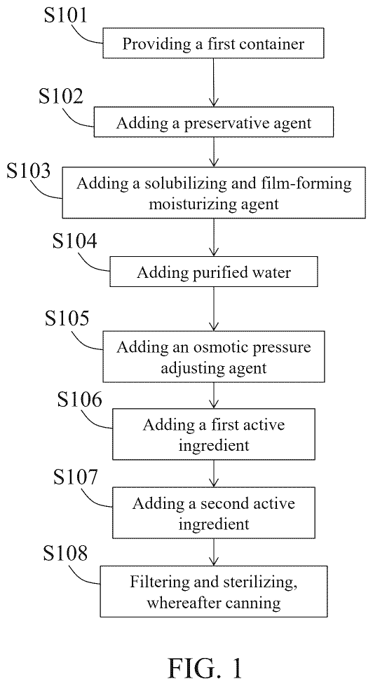

First, please refer to FIG. 1, which is a flowchart of a method for manufacturing a dressing that promotes wound healing according to the first embodiment of the present disclosure. First, in step S101, a first container is provided. Then, in step S102, a preservative agent is added into the first container, wherein the preservative agent accounts for not more than 0.3% by weight of the entire dressing. For example, but not limited to, the preservative agent accounts for 0.01, 0.05, 0.1, 0.15, 0.2, 0.25, or 0.3% by weight of the entire dressing. In this embodiment, the preservative agent is 1.2 grams of methylparaben (MP), but the present disclosure is not limited thereto. In addition, other types of preservatives can replace the MP. For example, Ethylparaben (EP) can be used instead. In other embodiments, the preservative agent can be MP or EP and accounts for 0.05 to 0.2% by weight of the entire dressing.

Thereafter, in step S103, a solubilizing and film-forming moisturizing agent is added into the first container, wherein the solubilizing and film-forming moisturizing agent accounts for not more than 8% by weight of the entire dressing. For example, but not limited to, the solubilizing and film-forming moisturizing agent accounts for 1, 2, 3, 4, 5, 6, 7 or 8% by weight of the entire dressing. The solubilizing and film-forming moisturizing agent can comprise at least one of propylene glycol (PG), glycerin, dipropylene glycol (DPG), and 1,3-propanediol (PDO). For example, the solubilizing and film-forming moisturizing agent consists essentially of PG and glycerin, wherein the PG accounts for 1 to 3% by weight of the entire dressing, and the glycerin accounts for 1 to 3% by weight of the entire dressing.

In this embodiment, the details of step S103 are as illustrated follows. First, a total of 20 grams of propylene glycol (PG), as a component of the solubilizing and film-forming moisturizing agent, is added into the first container. The mixture is appropriately stirred to dissolve the MP (preservative agent). Next, a total of 30 grams of glycerin, another component of the solubilizing and film-forming moisturizing agent, is further added into the first container, and the mixture is stirred uniformly. It should be noted here that the action of stirring is not a limiting requirement of the present disclosure. The stirring is performed solely to accelerate dissolution and uniform dispersion.

Thereafter, in step S104, purified water is added into the first container. Further, in this embodiment, the details of step S104 are described as follows. A total of 914 grams of reverse osmosis (RO) water is added into the first container, and the mixture is stirred uniformly. It should be noted here that the action of stirring is not a limiting requirement of the present disclosure. The stirring is performed solely to accelerate dissolution and uniform dispersion.

Thereafter, in step S105, an osmotic pressure adjusting agent is added into the first container. The osmotic pressure adjusting agent is selected from chloride salts, for example, sodium chloride, and accounts for 0.4 to 1% by weight of the entire dressing. Furthermore, the osmotic pressure adjusting agent accounts for, for example but not limited to, 0.4, 0.5, 0.6, 0.7, 0.8, 0.9, or 1% by weight of the entire dressing. It should be noted that the present disclosure is not limited to the osmotic pressure adjusting agent. The osmotic pressure adjusting agent can also be selected from at least one of chloride salts and lactate salts. Further, in this embodiment, the details of step S105 are described as follows. A total of 9 grams of the osmotic pressure adjusting agent, namely sodium chloride, is added into the first container, and the mixture is stirred uniformly. It should be noted here that the action of stirring is not a limiting requirement of the present disclosure. The stirring is performed solely to accelerate dissolution and uniform dispersion.

Furthermore, it should be noted that the dressing can be adjusted to be hypotonic, isotonic, or hypertonic. The present disclosure is not limited to the dressing in hypotonic, isotonic, or hypertonic state. Preferably, the dressing is adjusted to be hypertonic, thereby allowing undesirable tissue fluid from the wound of an organism to drain.

Thereafter, in step S106, a first active ingredient is added into the first container, wherein the first active ingredient is low-molecular-weight hyaluronic acid (HA), the molecular weight of the low-molecular-weight HA is not more than 500,000 Daltons, and the low-molecular-weight HA accounts for 0.01% to 1% by weight of the entire dressing. For example, but not limited to, the low-molecular-weight HA accounts for 0.01%, 0.02%, 0.03%, 0.04%, 0.05%, 0.1%, 0.2%, 0.3%, 0.4%, 0.5%, 0.6%, 0.7%, 0.8%, 0.9%, or 1% by weight of the entire dressing. Furthermore, in this embodiment, the details of step S106 are depicted as follows. A total of 2 grams of the low-molecular-weight HA is added into the first container, and the mixture is stirred uniformly. It should be noted here that the action of stirring is not a limiting requirement of the present disclosure. The stirring is performed solely to accelerate dissolution and uniform dispersion.

Thereafter, in step S107, a second active ingredient is added into the first container, wherein the second active ingredient is a plant stem cell extract and accounts for 0.1% to 5% by weight of the entire dressing. For example, but not limited to, the plant stem cell extract accounts for 0.1%, 0.2%, 0.3%, 0.4%, 0.5%, 1%, 2%, 3%, 4%, or 5% by weight of the entire dressing. Furthermore, in this embodiment, the details of step S107 are described as follows. A total of 5 grams of the plant stem cell extract is added into the first container, and the mixture is stirred uniformly. It should be noted here that the action of stirring is not a limiting requirement of the present disclosure. The stirring is performed solely to accelerate dissolution and uniform dispersion.

In this embodiment, the plant stem cell extract is an Orchidaceae plant stem cell extract. However, the present disclosure is not limited thereto. Further, the method for obtaining the plant stem cell extract is described as follows. Under aseptic conditions, plant tissue is cut, and plant stem cell mass is cultured using a plant tissue culture technique. A specific weight (e.g., 1 gram) of the plant stem cell mass in an aseptic state is taken, and an appropriate amount of liquid nitrogen is added into a mortar and rapidly ground into a powder form. After that, a specific volume (e.g., 1 mL) of sterile distilled water is added into the mortar and ground again. All substances in the mortar are aspirated into a centrifuge tube of a specific volume (e.g., 15 mL). After rapid shaking for a specific period of time (e.g., 3 minutes) and thorough mixing, centrifugation is performed. During centrifugation, the temperature is set to a low temperature (e.g., 4 degrees Celsius), the rotation speed is set to several thousand to tens of thousands of rpm (e.g., 10,000 rpm), and the centrifugation time is set to several minutes (e.g., 3 minutes). Next, the supernatant in the centrifuge tube is taken into another new centrifuge tube, and then aspirated with a syringe needle so that the supernatant passes through a filter membrane with a pore size of 0.1 to 0.5 micrometers (e.g., a 0.22 micrometer filter membrane), and then collected in an Eppendorf tube for preservation. The liquid in the Eppendorf tube is the concentrated stock solution of the above-mentioned plant stem cell extract. The concentrated stock solution is diluted by several thousand to tens of thousands of times to be used as the plant stem cell extract, so as to avoid cell damage. Therefore, the exact value of the weight percentage of the above-mentioned plant stem cell extract of the entire dressing is related to the dilution factor, and its percentage accounts for 0.1% to 5% by weight of the entire dressing depending on the dilution situation.

Thereafter, in step S108, because the dressing is in an aqueous liquid form, the liquid in the first container can be filtered and sterilized, whereafter it is canned. The filtration can be performed using a filter membrane with a pore size of 0.1 to 0.5 micrometers, for example, but not limited to, using a 0.22 micrometer filter membrane.

It should be noted here that the sequence of the above-mentioned steps S102 to S107 is not intended to limit the present disclosure, and the sequence can be adjusted. Furthermore, at least one of the above-mentioned steps S102, S103, S105, and S108 may be removed according to requirements and actual conditions. In addition, between steps S107 and S108, an additional step of adding purified water into the first container to adjust the total weight of the dressing may be performed. For example, the total weight of the entire dressing can be adjusted to 1,000 grams.

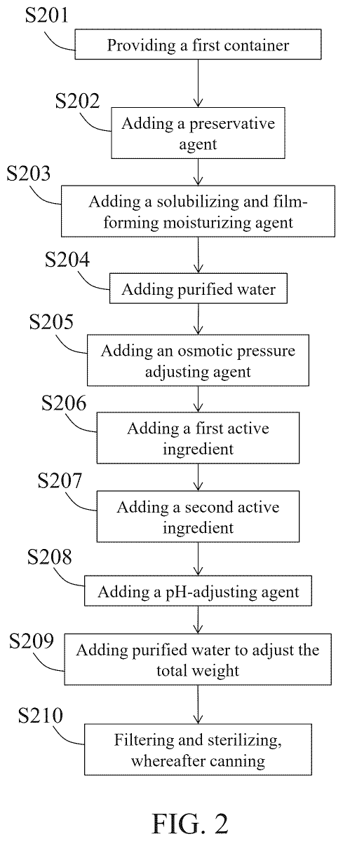

Please refer to FIG. 2, which is a flow chart of a method for manufacturing the dressing that promotes wound healing according to a second embodiment of the present disclosure. First, in step S201, a first container is provided. Then, in step S202, a preservative agent is added into the first container, wherein the preservative agent accounts for not more than 0.3% by weight of the entire dressing. In this embodiment, the preservative agent is 1.2 grams of MP.

Thereafter, in step S203, a solubilizing and film-forming moisturizing agent is added into the first container, wherein the solubilizing and film-forming moisturizing agent accounts for not more than 8% by weight of the entire dressing. In this embodiment, the details of step S203 are described as follows. First, a total of 20 grams of propylene glycol (PG), as a component of the solubilizing and film-forming moisturizing agent, is added into the first container. The mixture is appropriately stirred to dissolve the MP (preservative agent). Next, a total of 30 grams of glycerin, as another component of the solubilizing and film-forming moisturizing agent, is further added into the first container, and the mixture is stirred uniformly. It should be noted here that the action of stirring is not a limiting requirement of the present disclosure. The stirring is performed solely to accelerate dissolution and uniform dispersion.

Next, in step S204, purified water is added into the first container. Furthermore, in this embodiment, the details of step S204 are illustrated as follows. A total of 850 grams of RO water is added into the first container, and the mixture is stirred uniformly. It should be noted here that the action of stirring is not a limiting requirement of the present disclosure. The stirring is performed solely to accelerate dissolution and uniform dispersion.

Then, in step S205, an osmotic pressure adjusting agent is added into the first container, wherein the osmotic pressure adjusting agent is selected from at least one of chloride salts and lactate salts and accounts for 0.4% to 1% by weight of the entire dressing. In this embodiment, the details of step S205 are depicted as follows. A total of 9 grams of the osmotic pressure adjusting agent, which is sodium chloride, is added into the first container, and the mixture is stirred uniformly. Furthermore, as mentioned above, the action of stirring is not a limiting requirement of the present disclosure.

Thereafter, in step S206, a first active ingredient is added into the first container, wherein the first active ingredient is low-molecular-weight HA, the molecular weight of the low-molecular-weight HA is not more than 500,000 Daltons, and the low-molecular-weight HA accounts for 0.01% to 1% by weight of the entire dressing. In this embodiment, the details of step S206 are described as follows. A total of 3.2 grams of the low-molecular-weight HA is added into the first container, and the mixture is stirred uniformly. Furthermore, as mentioned above, the action of stirring is not a limiting requirement of the present disclosure.

Thereafter, in step S207, a second active ingredient is added into the first container, wherein the second active ingredient is β-nicotinamide mononucleotide (β-NMN) and accounts for 0.01% to 1% by weight of the entire dressing. For example, but not limited to, the β-NMN accounts for 0.01%, 0.02%, 0.03% or up to 1%. In this embodiment, the details of step S207 are described as follows. A total of 1 gram of the β-NMN is added into the first container, and the mixture is stirred uniformly. Furthermore, as mentioned above, the action of stirring is not a limiting requirement of the present disclosure.

Thereafter, in step S208, a pH-adjusting agent is added into the first container to adjust the pH of the dressing to a value from 5.5 to 9.0. For example, the pH of the dressing is adjusted to 5.5, 6, 6.5, 7, 7.5, 8, 8.5, or 9. In addition, the pH-adjusting agent can be phosphates. The phosphates can be, for example, but not limited to, at least one of potassium dihydrogen phosphate (KH₂PO₄) and disodium hydrogen phosphate (Na₂HPO₄). However, the present disclosure is not limited by the type of pH-adjusting agent. In this embodiment, potassium dihydrogen phosphate and disodium hydrogen phosphate are added to adjust pH of the dressing to 6.5.

Thereafter, in step S209, purified water is added into the first container to adjust the total weight of the dressing. For example, the total weight of the dressing is adjusted to 1,000 grams. Next, in step S210, because the dressing is in an aqueous liquid form, the liquid in the first container can be filtered and sterilized before canning. The filtration can be performed using a filter membrane with a pore size of 0.1 to 0.5 micrometers (μm), for example, but not limited to, using a filter membrane with a pore size of 0.22 μm.

It should be noted here that the sequence of the above-mentioned steps S202 to S207 is not intended to limit the present disclosure and can be adjusted. Furthermore, at least one of the above-mentioned steps S202, S203, S205, S208, S209, and S210 may be removed according to requirements and actual conditions.

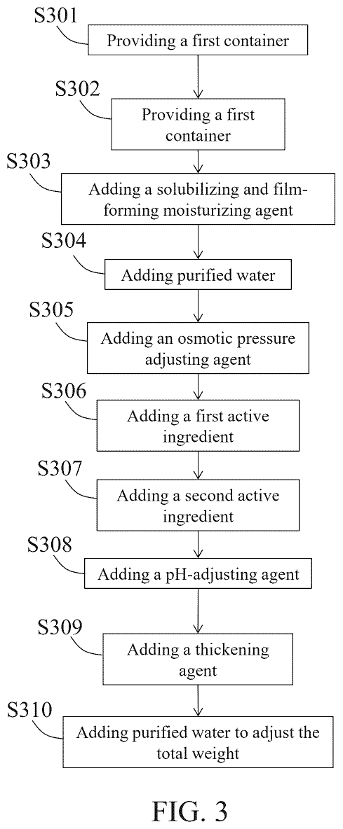

Please refer to FIG. 3, which is a flow chart of a method for manufacturing a dressing that promotes wound healing according to a third embodiment of the present disclosure. First, in step S301, a first container is provided. Then, in step S302, a preservative agent is added into the first container, wherein the preservative agent accounts for not more than 0.3% by weight of the entire dressing. In this embodiment, the preservative agent is 2 grams of MP.

Thereafter, in step S303, a solubilizing and film-forming moisturizing agent is added into the first container, wherein the solubilizing and film-forming moisturizing agent accounts for not more than 8% by weight of the entire dressing. In this embodiment, the details of step S303 are described as follows. First, a total of 30 grams of propylene glycol (PG), as a component of the solubilizing and film-forming moisturizing agent, is added into the first container, and the mixture is appropriately stirred to dissolve the MP (preservative agent). Then, a total of 30 grams of glycerin, as another component of the solubilizing and film-forming moisturizing agent, is added into the first container, and the mixture is stirred uniformly. Furthermore, as mentioned above, the action of stirring is not a limiting requirement of the present disclosure.

Thereafter, in step S304, purified water is added into the first container. Further, in this embodiment, the details of step S304 are described as follows. A total of 800 grams of reverse osmosis water at 40 to 50 degrees Celsius is added into the first container, and the mixture is stirred uniformly. Furthermore, as mentioned above, the action of stirring is not a limiting requirement of the present disclosure.

Thereafter, in step S305, an osmotic pressure adjusting agent is added into the first container, wherein the osmotic pressure adjusting agent is selected from at least one of chloride salts and lactate salts, and accounts for 0.4% to 1% by weight of the entire dressing. In this embodiment, the details of step S305 are described as follows. A total of 9 grams of the osmotic pressure adjusting agent, which is sodium chloride, is added into the first container, and the mixture is stirred uniformly. Furthermore, as mentioned above, the action of stirring is not a limiting requirement of the present disclosure.

Thereafter, in step S306, a first active ingredient is added to the first container, wherein the first active ingredient is low-molecular-weight HA, the molecular weight of the low-molecular-weight HA is not more than 500,000 Daltons, and the low-molecular-weight HA accounts for 0.01% to 1% by weight of the entire dressing. In this embodiment, the details of step S306 are described as follows. A total of 1.2 grams of the low-molecular-weight HA is added into the first container, and the mixture is stirred uniformly. Furthermore, as mentioned above, the action of stirring is not a limiting requirement of the present disclosure.

Thereafter, in step S307, a second active ingredient is added into the first container, wherein the second active ingredient is a plant stem cell extract and accounts for 0.1% to 5% by weight of the entire dressing. Further, in this embodiment, the details of step S307 are described as follows. A total of 2 grams of the plant stem cell extract is added into the first container, and the mixture is stirred uniformly. Furthermore, as mentioned above, the action of stirring is not a limiting requirement of the present disclosure. In addition, the method for obtaining plant stem cell extract is as described above.

Next, in step S308, a pH-adjusting agent is added into the first container to adjust the pH of the dressing to a value from 5.5 to 9. In this embodiment, potassium dihydrogen phosphate and disodium hydrogen phosphate are added into the first container to adjust the pH of the dressing to 6.6.

Thereafter, in step S309, a thickening agent is added into the first container to render the dressing in a gel form. The thickening agent is selected from the group consisting of Xanthan gum, hydroxyethylcellulose (HEC) gum, high-molecular-weight HA, and a combination thereof, but the present disclosure is not limited thereto. In this embodiment, the details of step S309 are described as follows. A total of 3.00 grams of the HEC gum is added into the first container, the mixture is thoroughly stirred to render the dressing in a gel form, and cooled to room temperature.

Thereafter, in step S310, purified water is added into the first container to adjust the total weight of the dressing. For example, the total weight of the dressing is adjusted to 1,000 grams. After step S310, the gel-form dressing can be further canned.

It should be noted here that the sequence of the above-mentioned steps S302 to S307 is not intended to limit the present disclosure and can be adjusted. Furthermore, at least one of the above-mentioned steps S302, S303, S305, S308, and S310 may be removed according to requirements and actual conditions.

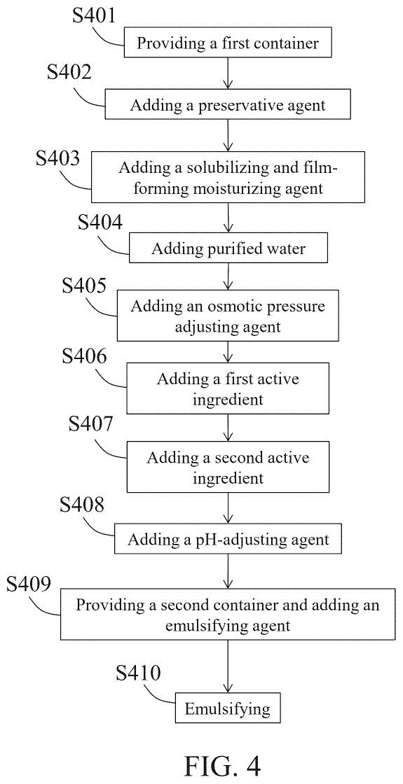

Please refer to FIG. 4, which is a flow chart of a method for manufacturing a dressing that promotes wound healing according to a fourth embodiment of the present disclosure. First, in step S401, a first container is provided. Then, in step S402, a preservative agent is added into the first container, wherein the preservative agent accounts for not more than 0.3% by weight of the entire dressing. In this embodiment, the preservative agent is 2 grams of MP.

Thereafter, in step S403, a solubilizing and film-forming moisturizing agent is added into the first container, wherein the solubilizing and film-forming moisturizing agent accounts for not more than 8% by weight of the entire dressing. In this embodiment, the details of step S403 are described as follows. First, a total of 30 grams of PG, as a component of the solubilizing and film-forming moisturizing agent, is added into the first container, and the mixture is appropriately stirred to dissolve the preservative agent, namely MP. Then, a total of 30 grams of glycerin, as another component of the solubilizing and film-forming moisturizing agent, is added into the first container, and the mixture is stirred uniformly. Furthermore, as mentioned above, the action of stirring is not a limiting requirement of the present disclosure.

Thereafter, in step S404, purified water is added into the first container. Further, in this embodiment, the details of step S404 are described as follows. A total of 800 grams of reverse osmosis water at 40-50 degrees Celsius is added into the first container, and the mixture is stirred uniformly. Furthermore, as mentioned above, the action of stirring is not a limiting requirement of the present disclosure.

Then, in step S405, an osmotic pressure adjusting agent is added into the first container, wherein the osmotic pressure adjusting agent is selected from at least one of chloride salts and lactate salts, and accounts for 0.4% to 1% by weight of the entire dressing. In this embodiment, the details of step S405 are described as follows. A total of 6 grams of the osmotic pressure adjusting agent, which is sodium chloride, is added into the first container, and the mixture is stirred uniformly. Furthermore, as mentioned above, the action of stirring is not a limiting requirement of the present disclosure.

Next, in step S406, a first active ingredient is added into the first container, wherein the first active ingredient is low-molecular-weight HA, the molecular weight of the low-molecular-weight HA is not more than 500,000 Daltons, and the low-molecular-weight HA accounts for 0.01% to 1% by weight of the entire dressing. In this embodiment, the details of step S406 are described as follows. A total of 0.2 grams of low-molecular-weight HA is added into the first container, and the mixture is stirred uniformly. Furthermore, as mentioned above, the action of stirring is not a limiting requirement of the present disclosure.

Thereafter, in step S407, a second active ingredient is added into the first container, wherein the second active ingredient is β-nicotinamide mononucleotide and accounts for 0.01% to 1% by weight of the entire dressing. Further, in this embodiment, the details of step S407 are described as follows. A total of 0.2 grams of the β-nicotinamide mononucleotide is added into the first container, and the mixture is stirred uniformly. Furthermore, as mentioned above, the action of stirring is not a limiting requirement of the present disclosure.

Thereafter, in step S408, a pH-adjusting agent is added into the first container to adjust the pH of the dressing to a value between 5.5 and 9. In this embodiment, potassium dihydrogen phosphate and disodium hydrogen phosphate are added into the first container to adjust the pH of the dressing to 6.6.

Thereafter, in step S409, a second container is provided, and an emulsifying agent is added into the second container. The emulsifying agent is for rendering the dressing in an emulsion form, and is selected from the group consisting of polysorbate (TWEEN), sorbitan laurate (SPAN), mineral oil, vegetable oil, and a combination thereof. In this embodiment, the details of step S409 are described as follows. First, a total of 12.6 grams of the TWEEN and 17.4 grams of the SPAN are added into the second container. Then, a total of 20 grams of the mineral oil is added into the second container.

Then, in step S410, the liquid from the first container containing the aqueous liquid-form dressing is transferred into the second container that comprises an emulsifying agent to render the dressing in an emulsion form. Further, in this embodiment, the details of step S410 are described as follows. An emulsification device is turned on, and purified water is added into the second container to adjust the total weight of the emulsion-form dressing to 1,000 grams. The emulsification speed of the emulsification device is set to 2,000 to 3,000 rpm, and the emulsification time is set to 10 to 15 minutes. The method further comprises the step of canning the emulsion-form dressing after step S410.

It should be noted here that the sequence of the above-mentioned steps S402 to S407 is not intended to limit the present disclosure and can be adjusted. Furthermore, at least one of the above-mentioned steps S402, S403, S405, and S408 may be removed according to requirements and actual conditions. Furthermore, in step S410, purified water may optionally not be added to adjust the total weight of the dressing.

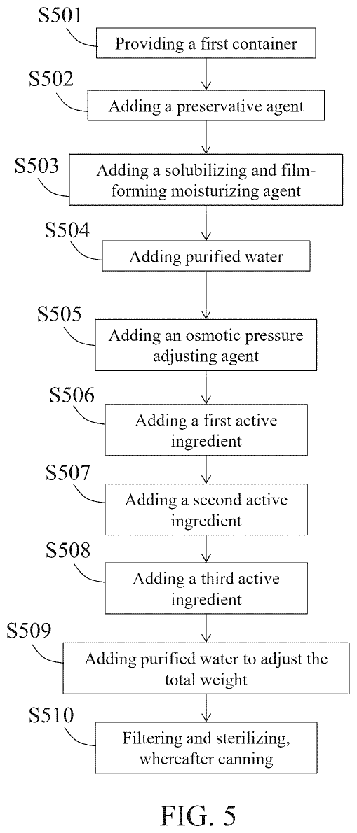

Please refer to FIG. 5, which is a flow chart of a method for manufacturing a dressing that promotes wound healing according to a fifth embodiment of the present disclosure. First, in step S501, a first container is provided. Then, in step S502, a preservative agent is added into the first container, wherein the preservative agent accounts for not more than 0.3% by weight of the entire dressing. In this embodiment, the preservative agent is 1.2 grams of MP.

Then, in step S503, a solubilizing and film-forming moisturizing agent is added into the first container, wherein the solubilizing and film-forming moisturizing agent accounts for not more than 8% by weight of the entire dressing. In this embodiment, the details of step S503 are described as follows. First, a total of 20 grams of PG, as a component of the solubilizing and film-forming moisturizing agent, is added into the first container, and the mixture is appropriately stirred to dissolve the MP (preservative agent). Then, a total of 30 grams of glycerin, as another component of the solubilizing and film-forming moisturizing agent, is further added into the first container, and the mixture is stirred uniformly. Furthermore, as mentioned above, the action of stirring is not a limiting requirement of the present disclosure.

Thereafter, in step S504, purified water is added into the first container. Further, in this embodiment, the details of step S504 are described as follows. A total of 914 grams of reverse osmosis water is added into the first container, and the mixture is stirred uniformly. Furthermore, as mentioned above, the action of stirring is not a limiting requirement of the present disclosure.

Thereafter, in step S505, an osmotic pressure adjusting agent is added into the first container, wherein the osmotic pressure adjusting agent is selected from at least one of chloride salts and lactate salts, and accounts for 0.4% to 1% by weight of the entire dressing. In this embodiment, the details of step S505 are described as follows. A total of 9 grams of the osmotic pressure adjusting agent, which is sodium chloride, is added into the first container, and the mixture is stirred uniformly. Furthermore, as mentioned above, the action of stirring is not a limiting requirement of the present disclosure.

Thereafter, in step S506, a first active ingredient is added into the first container, wherein the first active ingredient is low-molecular-weight HA, the molecular weight of the low-molecular-weight HA is not more than 500,000 Daltons and the low-molecular-weight HA accounts for 0.01% to 1% by weight of the entire dressing. In this embodiment, the details of step S506 are described as follows. A total of 2 grams of low-molecular-weight HA is added into the first container, and the mixture is stirred uniformly. Furthermore, as mentioned above, the action of stirring is not a limiting requirement of the present disclosure.

Thereafter, in step S507, a second active ingredient is added into the first container, wherein the second active ingredient is β-nicotinamide mononucleotide and accounts for 0.01% to 1% by weight of the entire dressing. Further, in this embodiment, the details of step S507 are described as follows. A total of 2 grams of β-nicotinamide mononucleotide is added into the first container, and the mixture is stirred uniformly. Furthermore, as mentioned above, the action of stirring is not a limiting requirement of the present disclosure.

Thereafter, in step S508, a third active ingredient is added into the first container, wherein the third active ingredient is a plant stem cell extract and accounts for not more than 5% by weight of the entire dressing. Further, in this embodiment, the details of step S508 are described as follows. A total of 4 grams of the plant stem cell extract is added into the first container, and the mixture is stirred uniformly. Furthermore, as mentioned above, the action of stirring is not a limiting requirement of the present disclosure. In addition, the method for obtaining the plant stem cell extract is as described above.

Thereafter, in step S509, purified water is added into the first container to adjust the total weight of the dressing, and the mixture is stirred uniformly. For example, the total weight of the dressing is adjusted to 1,000 grams. Furthermore, as mentioned above, the action of stirring is not a limiting requirement of the present disclosure. Next, in step S510, since the dressing is in an aqueous liquid form, the liquid in the first container can be filtered and sterilized, whereafter it is canned. Filtration can be performed using a filter membrane with a pore size of 0.1 to 0.5 micrometers, for example, but not limited to, a filter membrane with a pore size of 0.22 micrometers.

It should be noted here that the sequence of the above-mentioned steps S502 to S508 is not intended to limit the present disclosure and can be adjusted. Furthermore, at least one of the above-mentioned steps S502, S503, S505, S509, and S510 may be removed according to requirements and actual conditions.

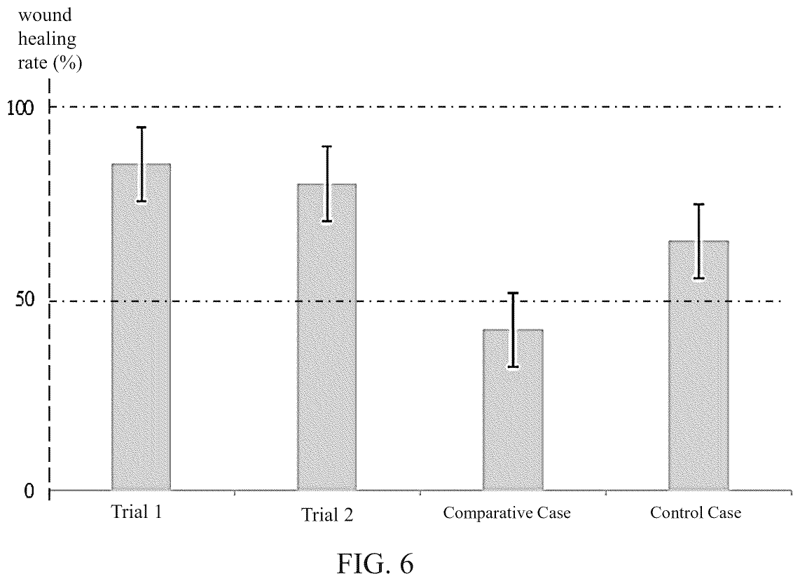

Please refer to FIG. 6, which shows a histogram of wound healing rate in a cell experiment using the dressing that promotes wound healing provided by the present disclosure. In this cell experiment, the mouse 3T3-L1 cell line was used to assess wound healing. Mouse 3T3-L1 cells are fibroblasts derived from mouse embryos and are a widely used in vitro model for studying Adipogenesis (formation of adipocytes), obesity, and metabolic diseases. Cells were cultured in Durbeco Modified Eagle Medium (DMEM) supplemented with fetal bovine serum (FBS) and seeded in 6-well cell culture dishes at a density of 1×10⁶ cells/well, and cultured for 24 hours to facilitate attachment and monolayer formation. Subsequently, under sterile conditions, linear scratches were made on the cell monolayer using the tip of a 200 μL dropper to simulate wounds. If necessary, the cells were gently rinsed with sterile fetal bovine serum to remove airborne cells and debris generated by the scratches. The solution was then replaced with culture medium containing the test dressing or physiological saline, and the culture dish was returned to the incubator to maintain the culture conditions.

Trial 1 served as the first Experimental Group, and its culture medium contained the dressing from the first embodiment described above. Trial 2 served as the second Experimental Group, and its culture medium contained the dressing from the second embodiment described above. Comparative Case served as Comparison Group, and its culture medium contained a commercially available dressing. The commercially available dressing is an aqueous wound dressing designed to aid in the repair and regeneration of damaged tissue. Its ingredients comprised sterile water, hyaluronic acid sodium salt, buffer solution, stabilizers, preservatives, microencapsulated polysaccharide, and oligopeptide I. Control Case served as a blank Control Group, and its culture medium was physiological saline, i.e., no dressing was applied. After the scratches were established, the same scratch area was continuously imaged in a fixed field of view using an inverted microscope. The image acquisition time points included 0, 12, 24, and 48 hours for quantitative comparison.

All images were measured and analyzed using ImageJ software, and the scratch width (or area) at 0 hours was used as the baseline to calculate the wound healing rate at each time point, using the following formulas: (Y_0 HR−Y_12 HR)/Y_0 HR, (Y_12 HR−Y_24 HR)/0 Y_HR, and (Y_24 HR−Y_48 HR)/Y_0 HR, where Y_0 HR represents the scratch width (or area) at 0 hours, Y_12 HR represents the scratch width (or area) at 12 hours, Y_24 HR represents the scratch width (or area) at 24 hours, and Y_48 HR represents the scratch width (or area) at 48 hours. The above calculations represent the healing progress relative to the initial wound size within each time interval.

As shown in FIG. 6, the experimental results show that the quantitative data at 24 hours after the scratch treatment are the most significant. During the 24-hour experimental treatment, all three cases (Trial 1, Trial 2, and Comparative Case) showed overall healing effects. The wound disclosure rate of Trial 1 was 85±8%, that of Trial 2 was 80±10%, that of Comparative Case was 65±8%, and that of Control Case was 42±6%. Among these, the p-values for Trial 1 and Trial 2 compared to Control Case was less than 0.01, indicating that the results were statistically significant.

Compared to Control Case, the above cell experiments confirmed that the dressings of Trial 1, Trial 2, and Comparative Case all exhibited faster wound healing speeds 24 hours after the cell lines were treated for wound healing. Furthermore, the healing completion rate of Trial 1 and Trial 2 rapidly approached completion within 24 hours, with Trial 1 performing slightly better than Trial 2, and its wound healing rate (or wound healing area) was at least twice that of Control Case. The dressings of Trial 1 and Trial 2 demonstrated wound healing effects and showed superior wound healing performance compared to the dressing of Comparative Case.

In addition to the cell experiments described above, related animal experiments were also conducted on the dressing provided by the present disclosure. The details of animal experiments are illustrated as follows.

Twenty-four thirteen-week-old male Wistar rats, sourced from LASCO Biotechnology Co., Ltd. (LASCO, Taiwan), were used in the animal experiments. All surgical procedures were performed under sterile conditions. Four 1 cm² full-thickness skin defects were created on the back of each rat, and they were randomly assigned to Control Case, Comparative Case, Trial 1, and Trial 2 according to the experimental design. Immediately after surgery, the wounds were treated according to their respective groups and covered with appropriate dressings.

Furthermore, Trial 1 served as the first Experimental Group, and its wounds were treated with the dressing described in the first embodiment. Trial 2 served as the second Experimental Group, and its wounds were treated with the dressing described in the second embodiment. Comparative Case served as Comparison Group, and its wounds were treated with the commercially available dressing. Control Case served as Control Group, i.e., the blank control group, and its wounds were treated with physiological saline.

In addition, in the present disclosure, after anesthetizing the rats, the wound area was first marked on the back with a marker, and then four 1 cm² full-thickness skin defects were created at the marked locations as a skin trauma model. This model can simulate clinical full-thickness trauma and can be used to evaluate the impact of different subsequent treatments on skin repair.

To compare the healing differences among Trial 1, Trial 2, Comparative Case, and Control Case in a skin wound model, this study continuously tracked the subjects from the day of injury (Day 0) until the end of Day 14. The Ratio of Wound Area (RWA) was measured and analyzed, where the RWA is defined as the current wound area divided by the wound area on Day 0.

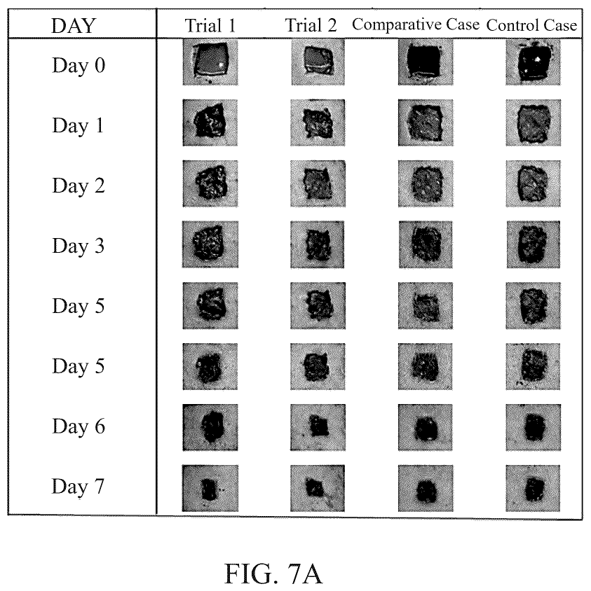

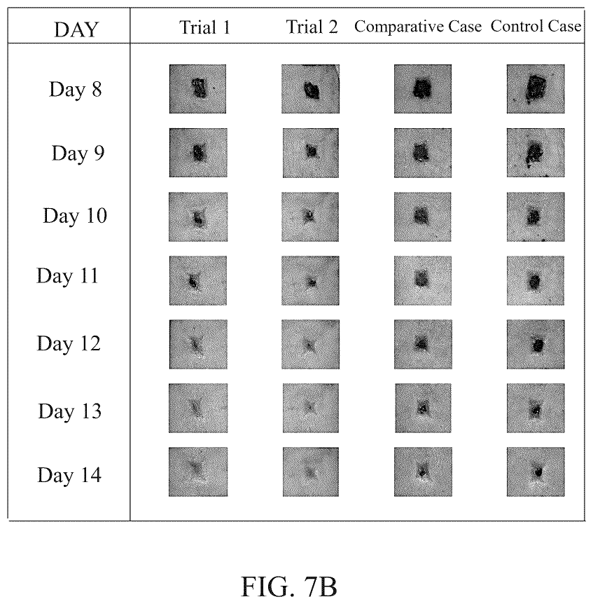

The wound area of each group (Trial 1, Trial 2, Comparative Case, and Control Case) was standardized to 1 on Day 0. The decrease in the wound area ratio overtime represents the gradual shrinkage and healing of the wound. The wound area ratios of each group (Trial 1, Trial 2, Comparative Case, and Control Case) from Day 0 to Day 14 are shown in Table 1 below, and the wound images from Day 0 to Day 14 are shown in FIGS. 7A and 7B.

TABLE 1

| | | | Comparative | Control |
|---|---|---|---|---|
| Day | Trial 1 | Trial 2 | Case | Case |
| Day 0 | 1.000 ± 0.000 | 1.000 ± 0.000 | 1.000 ± 0.000 | 1.000 ± 0.000 |
| Day 1 | 0.934 ± 0.040 | 1.013 ± 0.038 | 1.113 ± 0.040 | 1.075 ± 0.040 |
| Day 2 | 0.862 ± 0.031 | 0.957 ± 0.043 | 1.028 ± 0.047 | 0.978 ± 0.036 |
| Day 3 | 0.732 ± 0.029 | 0.854 ± 0.058 | 0.896 ± 0.053 | 0.882 ± 0.036 |
| Day 4 | 0.678 ± 0.028 | 0.754 ± 0.048 | 0.751 ± 0.049 | 0.751 ± 0.049 |
| Day 5 | 0.499 ± 0.042 | 0.550 ± 0.048 | 0.613 ± 0.070 | 0.614 ± 0.061 |
| Day 6 | 0.457 ± 0.039 | 0.514 ± 0.045 | 0.547 ± 0.051 | 0.560 ± 0.046 |
| Day 7 | 0.338 ± 0.034 | 0.429 ± 0.042 | 0.456 ± 0.054 | 0.470 ± 0.052 |
| Day 8 | 0.216 ± 0.025 | 0.303 ± 0.047 | 0.367 ± 0.063 | 0.335 ± 0.046 |
| Day 9 | 0.179 ± 0.017 | 0.240 ± 0.047 | 0.276 ± 0.048 | 0.224 ± 0.023 |
| Day 10 | 0.147 ± 0.016 | 0.195 ± 0.018 | 0.222 ± 0.045 | 0.193 ± 0.019 |
| Day 11 | 0.144 ± 0.009 | 0.185 ± 0.018 | 0.209 ± 0.040 | 0.172 ± 0.016 |
| Day 12 | 0.169 ± 0.015 | 0.198 ± 0.012 | 0.189 ± 0.036 | 0.165 ± 0.016 |
| Day 13 | 0.167 ± 0.013 | 0.189 ± 0.014 | 0.184 ± 0.032 | 0.151 ± 0.011 |
| Day 14 | 0.166 ± 0.015 | 0.180 ± 0.013 | 0.182 ± 0.030 | 0.163 ± 0.009 |

For ease of comparison, Control Case was used as the baseline at each time point. If the wound area ratio of Trial 1, Trial 2, and Comparative Case were lower than that of Control Case, it indicated better healing speed. Conversely, if the wound area ratio of Trial 1, Trial 2, and Comparative Case were higher than that of Control Case, it indicated relatively slow healing. The wound area ratios of Trial 1, Trial 2, Comparative Case, and Control Case all showed a decreasing trend over time, indicating that all animals entered progressive healing. However, the rate of decrease differed among different dressings, reflecting the different effects of each treatment on early contraction and epithelial coverage.

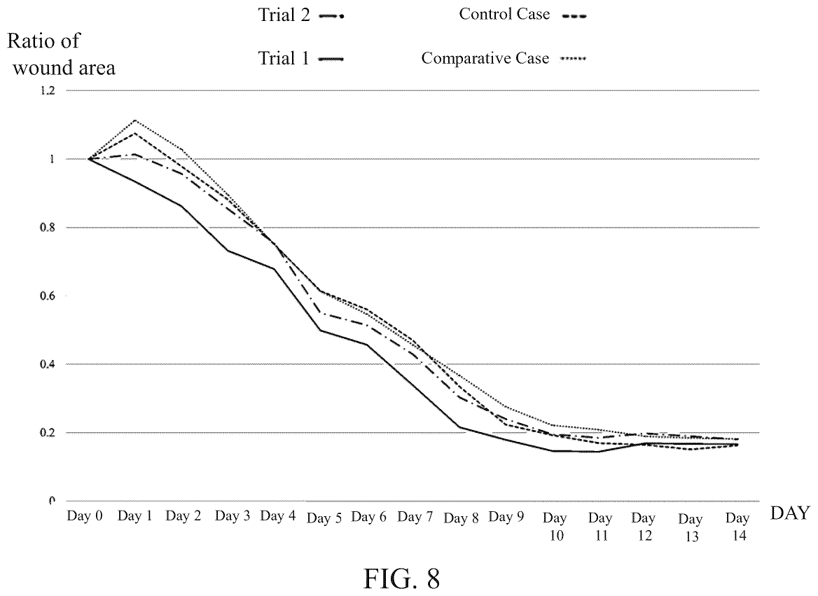

Please refer to FIG. 8, which is a line graph showing the ratio of wound area from Day 0 to Day 14 in animal experiments on wound healing using different dressings. The lines in the FIG. 8 are drawn based on the Table 1. In addition, for ease of observation, the vertical error indicators for each point in the line are omitted from the figure. From Day 1 to Day 3, the wound area ratio of Trial 1 was significantly lower than that of Comparative Case and Control Case on Day 1, indicating that the dressing of Trial 1 could promote wound contraction in the early stage of inflammation. The wound area ratios of Trial 2 and Comparative Case were similar to those of Control Case at this stage, showing no significant difference. From Day 1 to Day 3, the wound area ratios of Trial 1, Trial 2, Comparative Case, and Control Case all showed a decreasing trend over the days, indicating that all animals entered progressive healing. However, the rate of decrease varied among different dressings, reflecting the different effects of each treatment on early contraction, granulation tissue filling, and epithelial coverage.

From Day 4 to Day 7, the wound area ratios of Trial 1, Trial 2, Comparative Case, and Control Case all continued to decrease. Among them, the wound area ratio of Trial 1 remained at the lowest level, showing a sustained healing advantage. The wound area ratios of Trial 2 and Comparative Case were slightly lower than those of Control Case, but the difference was not as significant as that of Trial 1.

On Day 8, the wound area ratio of Trial 1 was significantly lower than that of Comparative Case and Control Case. Subsequently, the wound area ratios of Trial 1, Trial 2, Comparative Case, and Control Case all approached stable low values, with the differences gradually narrowing. On Day 14, the wound area ratios of Trial 1, Trial 2, Comparative Case, and Control Case all decreased to approximately 0.15 to 0.18, indicating almost complete wound healing. However, the wound area ratio of Trial 1 remained the lowest at most time points, indicating the fastest healing speed.

In summary, the dressing in Trial 1 demonstrated a sustained advantage over 14 days, significantly accelerating wound healing, particularly in the early stage. While the dressings in Trial 2 and Comparative Case also showed some promoting effect, their effects were more moderate compared to those in Trial 1. The decrease in the wound area ratio in Control Case primarily reflects the natural healing process.

In this animal experiment, to evaluate the impact of different dressing treatments on skin wound inflammation, Hematoxylin-Eosin staining was performed on the skin tissue in the wound area at weeks 1, 2, and 6 post-surgery, and the inflammation indices were calculated. Hematoxylin-Eosin staining is one of the most basic and widely used staining techniques in histology, embryology, and pathology, and is one of the gold standards for pathological diagnosis. Therefore, it will not be elaborated upon further.

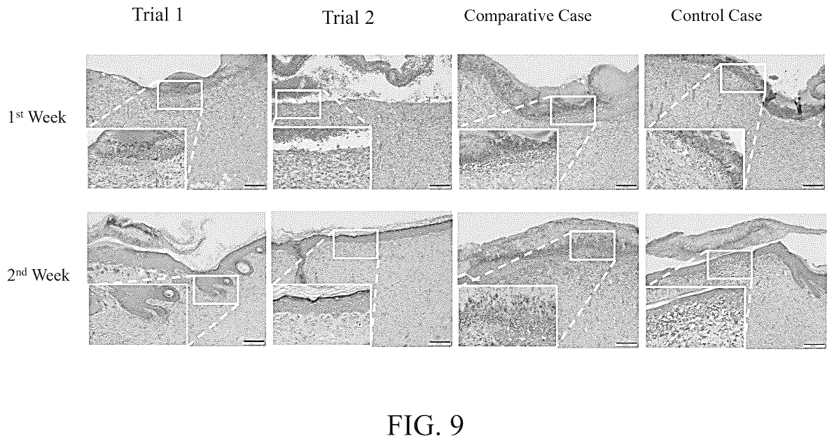

The interpretation of inflammatory response was based on microscopic histological observation, mainly including the degree of lymphocyte infiltration, vasodilation, and congestion. Evaluations were conducted based on these indicators. A score of 0 represents no inflammatory response, with no significant lymphocyte infiltration or vascular changes observed. A score of 1 represents mild inflammation, typically showing localized, sparse lymphocyte infiltration accompanied by slight vasodilation. A score of 2 represents moderate to severe inflammation, characterized by widespread lymphocyte infiltration accompanied by significant vasodilation. In this animal experiment, the above quantitative method was used to compare the effects of different dressing treatments on inflammatory response. Microscopic images of skin tissue at 1 and 2 weeks post-surgery in the above cases are shown in FIG. 9. A portion of each image in FIG. 9 was magnified to observe the inflammation. The inflammatory indices of skin tissue at postoperative Week 1, Week 2, and Week 6 in the above cases are shown in Table 2.

TABLE 2

| | | | Comparative | Control |
|---|---|---|---|---|
| Week | Trial 1 | Trial 2 | Case | Case |
| Week 1 | 1.67 ± 0.33 | 1.67 ± 0.33 | 1.67 ± 0.33 | 1.33 ± 0.33 |
| Week 2 | 0.33 ± 0.33 | 0.33 ± 0.33 | 1.33 ± 0.67 | 1.33 ± 0.67 |
| Week 6 | 0.00 ± 0.00 | 0.00 ± 0.00 | 0.00 ± 0.00 | 0.00 ± 0.00 |

As shown in Table 2, the inflammation indices of Trial 1, Trial 2, Comparative Case, and Control Case at Week 1 were 1.67±0.33, 1.67±0.33, 1.67±0.33, and 1.33±0.33, respectively. Overall, Trial 1, Trial 2, Comparative Case, and Control Case all showed high levels of inflammation. Obvious lymphocytic infiltration, vasodilation, and congestion were observed in the tissue sections, indicating that Week 1 postoperatively was the peak period of acute inflammation. Since all cases were in the early inflammatory phase at this time, the differences were not significant and were part of the normal repair process.

As shown in Table 2, the inflammation indices of Trial 1 and Trial 2 at Week 2 significantly decreased to 0.33±0.33, with only sporadic lymphocyte infiltration observed in their tissue sections. Vascular dilation and congestion were also significantly reduced, indicating that these two dressings effectively relieved inflammation in the intermediate stage. In contrast, the inflammation indices of Comparative Case and Control Case remained 1.33±0.67, with persistent lymphocyte aggregation and vasodilation still visible in their tissues, indicating that their inflammation had not been completely relieved.

As shown in Table 2, the inflammation indices at Week 6 in all cases decreased to 0.00±0.00, and significant lymphocytic infiltration, vasodilation, and congestion were no longer observed in tissue sections, indicating that the skin tissue had recovered to a non-inflammatory state at this stage. This also demonstrates that all experimental animals remained healthy under long-term observation and possessed normal self-repair and self-healing abilities.

In summary, the dressings used in Trial 1 and Trial 2 significantly reduce inflammation in the middle stage (Week 2) and are superior to the dressing used in Comparative Case and Control Case (without dressing). This indicates that the dressings used in Trial 1 and Trial 2 have potential application value in promoting inflammation relief in the early and middle stage of wound healing. In Week 1, as it is the peak inflammatory period, the minor differences among the cases are considered normal phenomena. By Week 6, all cases had ultimately recovered to a non-inflammatory state. Furthermore, it was confirmed that all experimental animals in the study were healthy and possessed self-healing abilities, and were able to complete tissue repair under long-term observation.

Furthermore, to evaluate the effect of different dressing treatments on epidermal layer repair in the skin wound model, the skin tissue from the wound area was subjected to Hematoxylin-Eosin staining at postoperative Weeks 1, 2, and 6, and the epidermal repair indices were calculated. The assessment of epidermal repair primarily involved observing whether the arrangement of keratinocytes was orderly and whether the layers were clearly distinct.

Through the microscope, stratum basale, stratum granulosum, stratum spinosum, and stratum corneum were completely identifiable. The ideal repair status is characterized by a uniform, intact stratum corneum without fragmentation or voids, with normal nuclear morphology and uniform staining, indicating normal epidermal differentiation and maintenance of a complete barrier function. The scoring criteria were defined as follows: a score of 0 indicates that no epidermal repair has occurred, 1 indicates that the epidermis has started to regenerate, and 2 indicates that the epidermal tissue is completely formed. The epidermal repair indices for the aforementioned cases at postoperative Weeks 1, 2, and 6 are shown in Table 3.

| | | | Comparative | Control |
|---|---|---|---|---|
| Week | Trial 1 | Trial 2 | Case | Case |
| Week 1 | 0.00 ± 0.00 | 0.00 ± 0.00 | 0.00 ± 0.00 | 0.00 ± 0.00 |
| Week 2 | 1.67 ± 0.33 | 2.00 ± 0.00 | 0.67 ± 0.67 | 0.00 ± 0.00 |
| Week 6 | 2.00 ± 0.00 | 2.00 ± 0.00 | 2.00 ± 0.00 | 1.67 ± 0.33 |

As shown in Table 3, Week 1 results indicate that the epidermal repair indices for all cases were 0.00±0.00, indicating that no nascent structures of the stratum corneum or organized cell arrangement were observed in the tissue sections.

As shown in Table 3, Week 2 results indicate that the epidermal repair indices for Trial 1 and Trial 2 reached 1.67±0.33 and 2.00±0.00, respectively, demonstrating that epidermal repair entered an active phase, with clearly discernible cell layers. However, the epidermal repair index for Comparative Case only reached 0.67±0.67, and the epidermal repair index for Control Case remained at 0.00±0.00, indicating that the repair progress was significantly delayed.

As shown in Table 3, Week 6 results indicate that the epidermal repair indices for Trial 1, Trial 2, and Comparative Case had all reached 2.00±0.00, demonstrating complete epidermal structure and uniform stratum corneum. However, the epidermal repair index for Control Case was 1.67±0.33, indicating that although recovery occurred, it was slightly lower than that of Trial 1, Trial 2, and Comparative Case.

In summary, the dressings used in Trial 1 and 2 allowed for noticeable epidermal repair in the skin tissue at the mid-stage. While Comparative Case and Control Case did not show significant epidermal repair at the mid-stage, they still ultimately achieved complete repair. Overall, the dressings used in Trial 1 and Trial 2 effectively accelerated the epidermal reconstruction process.

Furthermore, to evaluate the effect of different dressing treatments on dermal layer repair in the skin wound model, the skin tissue from the wound area was subjected to Hematoxylin and Eosin (H&E) staining at postoperative Weeks 1, 2, and 6, and the dermal repair indices were calculated. The assessment of dermal repair was primarily based on the arrangement of fibers and the state of the fibroblasts. Under microscopic observation, when the fibers appeared wavy and uniformly arranged, accompanied by a significantly increased number of plump fibroblasts and clear nuclei, it indicated that the dermal structure was gradually recovering and possessed good tension and supporting capacity. The scoring criteria are defined as follows: A score of 0 indicates that no dermal repair has occurred. A score of 1 indicates that the dermis has started to regenerate. A score of 2 indicates that the dermal tissue is completely formed. The dermal repair indices for the aforementioned cases at postoperative Weeks 1, 2, and 6 are shown in Table 4.

TABLE 4

| | | | Comparative | Control |
|---|---|---|---|---|
| Week | Trial 1 | Trial 2 | Case | Case |
| Week 1 | 0.00 ± 0.00 | 0.00 ± 0.00 | 0.00 ± 0.00 | 0.00 ± 0.00 |
| Week 2 | 0.33 ± 0.33 | 0.00 ± 0.00 | 0.00 ± 0.00 | 0.00 ± 0.00 |
| Week 6 | 1.00 ± 0.00 | 1.00 ± 0.00 | 1.00 ± 0.00 | 0.67 ± 0.33 |

As shown in Table 4, Week 1 results indicate that the dermal repair indices for all cases were 0.00±0.00, and no nascent fibers or dermal regeneration was observed in the tissue sections.

As shown in Table 4, Week 2 results indicate that the dermal repair index for Trial 1 was 0.33±0.33, with a small amount of fiber arrangement visible in the dermal layer, indicating mild dermal repair. The dermal repair indices for Trial 2, Comparative Case, and Control Case, however, remained at 0.00±0.00, with no nascent fibers or dermal regeneration observed in the tissue sections.

As shown in Table 4, Week 6 results indicate that the dermal repair indices for Trial 1, Trial 2, and Comparative Case all reached 1.00±0.00, with gradual regularization of dermal fibers visible in the sections. However, the dermal repair index for Control Case was 0.67±0.33, indicating a slightly lower degree of dermal repair.

In summary, the results show that the dressing in Trial 1 initiated dermal repair in the middle stage of the skin tissue repair process. The dressings in Trial 2 and Comparative Case gradually caught up in dermal repair in the late stage. Although the dermal layer of the skin tissue in Control Case eventually recovered, the extent was smaller. Overall, the dressing intervention in Trial 1 helps to accelerate dermal repair.

Furthermore, to evaluate the effect of different dressing treatments on angiogenesis in the skin wound, this animal experiment involved Hematoxylin-Eosin staining of the skin tissue from the wound area at postoperative Weeks 1, 2, and 6, and the angiogenesis repair indices were calculated. The assessment of angiogenesis focused on the number, distribution, and integrity of newly formed micro vessels, and whether the arrangement of endothelial cells was orderly and regular, and whether the lumen formation was clear. The formation of new blood vessels indicates the gradual recovery of local tissue oxygen and nutrient transport, reflecting metabolic demand and the activity of the regeneration process. A score of 0 indicates that no vascular repair has occurred. A score of 1 indicates that vessels have started to form. A score of 2 indicates that the vascular tissue is completely formed. The angiogenesis repair indices for the aforementioned cases at postoperative Weeks 1, 2, and 6 are shown in Table 5.

TABLE 5

| | | | Comparative | Control |
|---|---|---|---|---|
| Week | Trial 1 | Trial 2 | Case | Case |
| Week 1 | 0.00 ± 0.00 | 0.00 ± 0.00 | 0.00 ± 0.00 | 0.00 ± 0.00 |
| Week 2 | 1.00 ± 0.00 | 1.00 ± 0.00 | 0.67 ± 0.33 | 0.00 ± 0.00 |
| Week 6 | 1.33 ± 0.33 | 1.00 ± 0.00 | 1.33 ± 0.33 | 1.33 ± 0.33 |

As shown in Table 5, Week 1 results indicate that the angiogenesis repair indices for all the aforementioned cases were 0.00±0.00, indicating that no signs of vascular formation or angiogenesis were observed in the tissue sections.

As shown in Table 5, Week 2 results indicate that the angiogenesis repair indices for Trial 1 and Trial 2 both reached 1.00±0.00, demonstrating the presence of newly formed micro vessels in the sections. The angiogenesis repair index for Comparative Case was 0.67±0.33, indicating limited vascular recovery. In contrast, Control Case's angiogenesis repair index remained at 0.00±0.00, indicating no signs of vascular formation were observed in the sections.

As shown in Table 5, Week 6 results indicate that the angiogenesis repair indices for Trial 1 and Comparative Case were 1.33±0.33. The angiogenesis repair index for Trial 2 was 1.00±0.00, and the angiogenesis repair index for Control Case was 1.33±0.33, demonstrating that angiogenesis was significant in all cases at this time point.

In summary, the dressings of Trial 1 and Trial 2 can promote angiogenesis in the medium term. The dressing of Comparative Case gradually caught up in the late stage. Although Control Case showed no performance in the early stage, it eventually achieved a similar effect, indicating that self-healing capacity can restore angiogenesis in the long term.

Furthermore, to evaluate the effect of different dressing treatments on hair follicle regeneration in the skin wound, this animal experiment involved Hematoxylin-Eosin staining of the skin tissue from the wound area at postoperative Weeks 1, 2, and 6, and the hair follicle repair indices were calculated. Hair follicle regeneration was assessed based on the structural integrity and clarity of stratification within the Matrix, Papilla, and Bulb regions. Clear identification of the structures in the Matrix, Papilla, and Bulb indicates that the skin appendages possess regenerative potential, and suggests that the corresponding treatment has potential value in promoting deep repair and tissue reconstruction. The scoring criteria are defined as follows: A score of 0 indicates that no hair follicle regeneration has occurred. A score of 1 indicates that hair follicles have started to form. A score of 2 indicates that the hair follicle tissue is completely formed. The hair follicle repair indices for the aforementioned cases at postoperative Weeks 1, 2, and 6 are shown in Table 6.

TABLE 6

| | | | Comparative | Control |
|---|---|---|---|---|
| Week | Trial 1 | Trial 2 | Case | Case |
| Week 1 | 0.00 ± 0.00 | 0.00 ± 0.00 | 0.00 ± 0.00 | 0.00 ± 0.00 |
| Week 2 | 0.33 ± 0.33 | 0.67 ± 0.67 | 0.00 ± 0.00 | 0.00 ± 0.00 |
| Week 6 | 0.67 ± 0.67 | 1.33 ± 0.67 | 1.33 ± 0.67 | 1.33 ± 0.67 |

As shown in Table 6, Week 1 results indicate that the hair follicle repair indices for all the aforementioned cases were 0.00±0.00, indicating that no hair follicle structures were observed in the tissue sections.

As shown in Table 6, Week 2 results indicate that the hair follicle repair indices for Trial 1 and Trial 2 were 0.33±0.33 and 0.67±0.67, respectively, indicating that preliminary hair follicle regeneration had occurred. However, the hair follicle repair indices for Comparative Case and Control Case remained at 0.00±0.00, indicating that no hair follicle structures were observed in the tissue sections.

As shown in Table 6, Week 6 results indicate that the hair follicle repair indices for Trial 2 and Comparative Case both reached 1.33±0.67. The index for Control Case was also 1.33±0.67, demonstrating the recovery of the hair follicle bulb, papilla, and matrix structures in the sections. However, the hair follicle repair index for Trial 1 was 0.67±0.67, indicating a relatively lower degree of hair follicle regeneration.

In summary, the dressings of Trial 2 and Comparative Case showed better effects on hair follicle regeneration. The no-dressing treatment in Control Case was also able to achieve a similar level to the dressings of Trial 2 and Comparative Case in the late stage. The dressing of Trial 1 resulted in a slightly lower degree of hair follicle regeneration, indicating that hair follicle repair is a relatively late-stage phenomenon. In terms of hair follicle repair, the differences among the cases were not as significant as in the other aforementioned repair indicators.

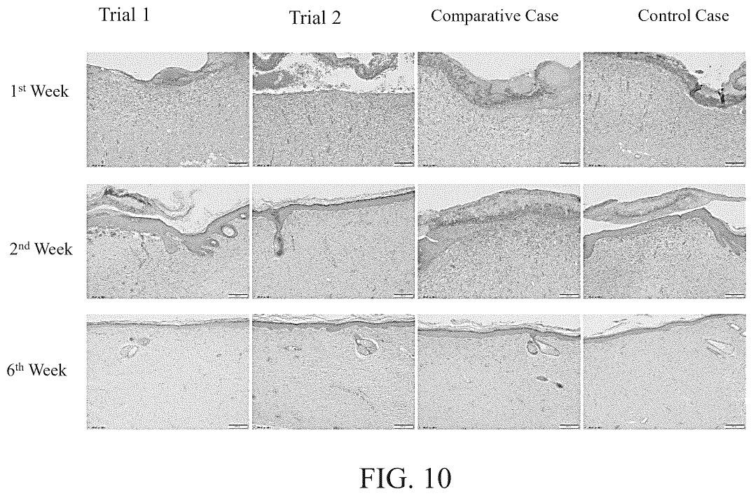

Furthermore, to evaluate the effect of different dressing treatments on the overall tissue repair in the skin wound, this animal experiment involved Hematoxylin-Eosin staining of the skin tissue from the wound area at postoperative Weeks 1, 2, and 6, and the overall repair indices were calculated. The scoring method for the overall repair index is the summation of scores from four aspects: epidermal layer recovery and proliferation, dermal layer recovery and proliferation, angiogenesis, and hair follicle regeneration. The significance of this index is the integration of multi-level repair indicators into a single quantitative value to comprehensively understand the overall healing status of the wound under different dressing treatments. This scoring method can eliminate the limitations of single-structure judgment, provide a more holistic evaluation criterion, and help identify the comprehensive effect of the dressings on skin regeneration and tissue reconstruction. Microscopic images of the skin tissue for the aforementioned cases at postoperative Weeks 1, 2, and 6 are shown in FIG. 10, and the overall repair indices for these cases at postoperative Weeks 1, 2, and 6 are shown in Table 7.

TABLE 7

| | | | Comparative | Control |
|---|---|---|---|---|
| Week | Trial 1 | Trial 2 | Case | Case |
| Week 1 | 0.00 ± 0.00 | 0.00 ± 0.00 | 0.00 ± 0.00 | 0.00 ± 0.00 |
| Week 2 | 3.33 ± 0.33 | 3.67 ± 0.67 | 1.33 ± 0.88 | 0.00 ± 0.00 |
| Week 6 | 5.00 ± 1.00 | 5.33 ± 0.67 | 5.67 ± 0.88 | 5.00 ± 1.53 |

As shown in Table 7, Week 1 results indicate that the overall repair indices for all the aforementioned cases were 0.00±0.00. The wound area in the tissue sections was primarily composed of inflammatory cell infiltration and tissue defects, with no nascent epidermal or dermal structures observed, indicating that the repair response had not yet been initiated.

As shown in Table 7, Week 2 results indicate that the overall repair indices for Trial 1 and Trial 2 increased to 3.33±0.33 and 3.67±0.67, respectively. This shows gradual coverage of the wound by the neo-epidermis and proliferation of fibroblasts in the dermal layer. The overall repair index for Comparative Case was only 1.33±0.88, indicating limited repair. In contrast, the overall repair index for Control Case remained at 0.00±0.00, demonstrating that the wound remained in a state of inflammation and tissue defect.

As shown in Table 7, Week 6 results indicate that all cases achieved significant repair. The overall repair indices for Trial 1, Trial 2, Comparative Case, and Control Case were 5.00±1.00, 5.33±0.67, 5.67±0.88, and 5.00±1.53, respectively. The sections showed a complete epidermal layer and a remodeled dermal layer, with fiber formation, angiogenesis, and hair follicle regeneration becoming increasingly apparent.

In summary, the dressings in Trial 1 and 2 showed significantly faster healing rates in the middle stage. While the dressing in Comparative Case progressed slowly initially, it eventually reached a similar level of healing. Control Case showed the weakest healing. Over the long term, all cases showed healing, but the use of external dressings accelerated healing, particularly the dressings in Trial 1 and Trial 2.

According to the above described contents, compared to the prior art, rigorous animal experiments have verified that the dressings provided by the present disclosure are of significant importance for improving clinical wound repair strategies. It has been confirmed that they can significantly reduce inflammation indices, accelerate epidermal coverage, and enhance overall repair during the early and medium stages of the healing timeline. This demonstrates their ability to promote a smooth transition from the inflammatory phase to the proliferative phase, providing a substantial advantage in the healing timeline.

This present disclosure is disclosed herein only by preferred embodiments. However, it should be understood by anyone skilled in the art that the above embodiments are for illustrative purposes only and are not intended to limit the scope of the patent rights claimed by this disclosure. All variations or substitutions equivalent to the above embodiments should be interpreted as being covered within the spirit or scope of the present disclosure. Therefore, the scope of protection of the present disclosure should be based on the claims defined below.

What is claimed is:

1. A dressing for promoting wound healing, used purified water as a solvent, comprising:
a first active ingredient, wherein the first active ingredient is low-molecular-weight hyaluronic acid (HA) having a molecular weight of not more than 500,000 Daltons and accounting for 0.01% to 1% by weight of the entire dressing; and
a second active ingredient, wherein the second active ingredient is β-nicotinamide mononucleotide (β-NMN) and accounts for 0.01% to 1% by weight of the entire dressing; or the second active ingredient is a plant stem cell extract and accounts for 0.1% to 5% by weight of the entire dressing.

2. The dressing for promoting wound healing according to claim 1, further comprising an osmotic pressure adjusting agent selected from one or more of chloride salts and lactate salts, which accounts for 0.4% to 1% by weight of the entire dressing.

3. The dressing for promoting wound healing according to claim 1, further comprising a solubilizing and film-forming moisturizing agent which accounts for not more than 8% by weight of the entire dressing.

4. The dressing for promoting wound healing according to claim 3, wherein the solubilizing and film-forming moisturizing agent comprises one or more of propylene glycol (PG), glycerin, dipropylene glycol (DPG), and 1,3-propanediol (PDO).

5. The dressing for promoting wound healing according to claim 4, wherein the solubilizing and film-forming moisturizing agent consists essentially of propylene glycol and glycerin, the propylene glycol accounts for 1% to 3% by weight of the entire dressing, and the glycerin accounts for 1% to 3% by weight of the entire dressing.

6. The dressing for promoting wound healing according to claim 1, further comprising a preservative agent accounting for not more than 0.3% by weight of the entire dressing.

7. The dressing for promoting wound healing according to claim 6, wherein the preservative agent is methylparaben (MP) or Ethylparaben (EP) and accounts for 0.05% to 0.2% by weight of the entire dressing.

8. The dressing for promoting wound healing according to claim 2, wherein the osmotic pressure adjusting agent is sodium chloride.

9. The dressing for promoting wound healing according to claim 1, wherein the second active ingredient is β-nicotinamide mononucleotide, and the dressing further comprises a third active ingredient, namely, a plant stem cell extract, which accounts for not more than 5% by weight of the entire dressing.

10. The dressing for promoting wound healing according to claim 1, wherein the plant stem cell extract is an orchid plant stem cell extract.

11. The dressing for promoting wound healing according to claim 1, further comprising a pH-adjusting agent for adjusting a pH value of the dressing to between 5.5 and 9.

12. The dressing for promoting wound healing according to claim 11, wherein the pH-adjusting agent is phosphates.

13. The dressing for promoting wound healing according to claim 1, wherein the dressing is in an aqueous liquid form.

14. The dressing for promoting wound healing according to claim 1, further comprising a thickening agent for rendering the dressing in a gel form.

15. The dressing for promoting wound healing according to claim 14, wherein the thickening agent is selected from the group consisting of xanthan gum, hydroxyethylcellulose (HEC) gum, high-molecular-weight hyaluronic acid, and combinations thereof.

16. The dressing for promoting wound healing according to claim 1, further comprising an emulsifying agent for rendering the dressing in an emulsion form.

17. The dressing for promoting wound healing according to claim 16, wherein the emulsifying agent is selected from the group consisting of polysorbate (TWEEN), sorbitan laurate (SPAN), mineral oil, vegetable oil, and combinations thereof.

18. A method for manufacturing a dressing that promotes wound healing, comprising steps of:
providing a first container;
adding purified water into the first container;
adding a first active ingredient into the first container, wherein the first active ingredient is low-molecular-weight hyaluronic acid having a molecular weight of not more than 500,000 Daltons and accounting for 0.01% to 1% by weight of the entire dressing; and
adding a second active ingredient into the first container, wherein the second active ingredient is β-nicotinamide mononucleotide and accounts for 0.01% to 1% by weight of the entire dressing, or the second active ingredient is a plant stem cell extract and accounts for 0.1% to 5% by weight of the entire dressing.

19. The method of claim 18, further comprising a step of: adding an osmotic pressure adjusting agent into the first container, wherein the osmotic pressure adjusting agent is selected from one or more of chloride salts and lactate salts and accounts for 0.4% to 1% by weight of the entire dressing.

20. The method of claim 18, further comprising a step of: adding a preservative agent into the first container, wherein the preservative agent accounts for not more than 0.3% by weight of the entire dressing.

21. The method of claim 18, further comprising a step of: adding a solubilizing and film-forming moisturizing agent into the first container, wherein the solubilizing and film-forming moisturizing agent accounts for not more than 8% by weight of the entire dressing.

22. The method of claim 18, wherein the dressing is in an aqueous liquid form, and the method further comprises a step of: filtering and sterilizing liquid in the first container, and then canning the liquid.

23. The method of claim 18, further comprising a step of: adding a pH-adjusting agent into the first container, and adjusting a pH value of the dressing to between 5.5 and 9.

24. The method of claim 18, wherein the second active ingredient is β-nicotinamide mononucleotide, and the method further comprises a step of: adding a third active ingredient into the first container, wherein the third active ingredient is a plant stem cell extract and accounts for not more than 5% by weight of the entire dressing.

25. The method of claim 18, further comprising a step of: adding a thickening agent into the first container to render the dressing in a gel form.

26. The method of claim 18, further comprising steps of:
adding an emulsifying agent into a second container;
transferring liquid in the first container into the second container; and
emulsifying mixture liquid in the second container to render the dressing in an emulsion form.
